(19) United States
(12) Patent Application Publication
(10) Pub. No.: US 2020/0023102 A1
(43) Pub. Date: Jan. 23, 2020

(54) WOUND THERAPY SYSTEM

(71) Applicant: Patrick Kenneth Powell, Farmington, MI (US)

(72) Inventor: Patrick Kenneth Powell, Farmington, MI (US)

(21) Appl. No.: 16/089,453

(22) PCT Filed: Apr. 5, 2017
(86) PCT No.: PCT/US2017/026083

§ 371 Date: Sep. 28, 2018

Related U.S. Application Data
(60) Provisional application No. 62/318,292, filed on Apr. 5, 2016.

(51) Int. Cl. A61M 1/00; A61F 13/02; A61F 13/00

(52) U.S. Cl. CPC A61M 1/009; A61F 13/0206; A61M 1/0066; A61M 2205/0288; A61F 13/00068; A61M 2205/0205; A61M 2205/0233; A61M 1/0035

(57) ABSTRACT

A wound therapy system according to an example of the present disclosure includes a wound therapy that has a porous body operable to carry a fluid from a wound site, a tube, and a pump connected to the porous body by the tube. The pump is operable to apply vacuum through the tube to draw fluid from the wound site.

### CROSS-REFERENCE TO RELATED APPLICATIONS

This application claims priority to U.S. Provisional Patent Application Ser. No. 62/318,292, filed Apr. 5, 2016.

### BACKGROUND

Wound dressings are typically used to cover and protect wounds during healing as well as protect surrounding healthy tissue from infection. Removing fluid from a wound site promotes healing of the wound and can reduce pain.

### SUMMARY

A wound therapy system according to an example of the present disclosure includes a wound therapy that has a porous body operable to carry a fluid from a wound site, a tube, and a pump connected to the porous body by the tube. The pump is operable to apply vacuum through the tube to draw fluid from the wound site.

A further embodiment of any of the foregoing embodiments include a chest wrap, the chest wrap configured to transform circumferential expansion and contraction of the chest wrap into energy for driving the pump.

In a further embodiment of any of the foregoing embodiments, the chest wrap further comprises at least one serpentine line that amplifies power generation from circumferential expansion and contraction of the chest wrap.

In a further embodiment of any of the foregoing embodiments, the porous body includes capillaries operable to carry the fluid through the porous body.

In a further embodiment of any of the foregoing embodiments, the porous body includes a plurality of segments.

In a further embodiment of any of the foregoing embodiments, the plurality of segments are irregular in size, shape, or both.

The wound therapy system of claim 5, further comprising a plurality of dividers separating the plurality of segments from one another.

In a further embodiment of any of the foregoing embodiments, the plurality of dividers are fluid-impermeable.

In a further embodiment of any of the foregoing embodiments, the plurality of dividers include at least one of an anti-bacterial and an anti-viral agent.

In a further embodiment of any of the foregoing embodiments, the porous body includes electrically conductive fibers.

A further embodiment of any of the foregoing embodiments include electrical leads operable to provide electrical impulses to the conductive fibers.

In a further embodiment of any of the foregoing embodiments, the electrical leads are connected to and draw power from the chest wrap.

In a further embodiment of any of the foregoing embodiments, the tube includes an absorbent material operable to absorb the fluid.

In a further embodiment of any of the foregoing embodiments, the absorbent material comprises a plurality of balls. The balls have an impermeable shell that has perforations, and an absorbent powder inside the impermeable shell.

In a further embodiment of any of the foregoing embodiments, the absorbent material solidifies upon contact with the fluid.

In a further embodiment of any of the foregoing embodiments, the wound therapy device further includes a plenum. The plenum in fluid communication with the tube.

A further embodiment of any of the foregoing embodiments include an adhesive dressing between the plenum and the porous body.

In a further embodiment of any of the foregoing embodiments, the adhesive dressing seals the wound site.

In a further embodiment of any of the foregoing embodiments, at least one of the plenum and adhesive dressing are magnetic, and the porous body is magnetic, and the plenum or adhesive surface and porous body attract and seal to one another.

A wound therapy system according to an example of the present disclosure includes a wound therapy device that has a foam wound dressing having a plurality of segments. The foam has capillaries operable to carry fluid from a wound site, a pneumatic tube in fluid communication with the wound therapy device, and a pump operable to apply vacuum to the foam via the pneumatic tube to draw fluid out of the wound site.

### BRIEF DESCRIPTION OF THE DRAWINGS

FIG. 1 schematically illustrates a wound therapy system on a patient.

FIG. 2 schematically illustrates the wound therapy system on a patient.

FIG. 3A schematically illustrates the wound therapy system.

FIG. 3B schematically illustrates the wound therapy system in more detail.

FIG. 4 schematically illustrates an absorbent material for a wound therapy system.

FIG. 5 schematically illustrates a pump for the wound therapy system.

FIG. 6 schematically illustrates a wound therapy device for the wound therapy system.

FIG. 7 schematically illustrates a wound therapy device for the wound therapy system in a wound site.

FIG. 8 schematically illustrates a segmented porous body for a wound therapy device.

FIG. 9 schematically illustrates an alternate segmented porous body for a wound therapy system.

The various features and advantages of the present disclosure will become apparent to those skilled in the art from the following detailed description. The drawings that accompany the detailed description can be briefly described as follows.

### DETAILED DESCRIPTION

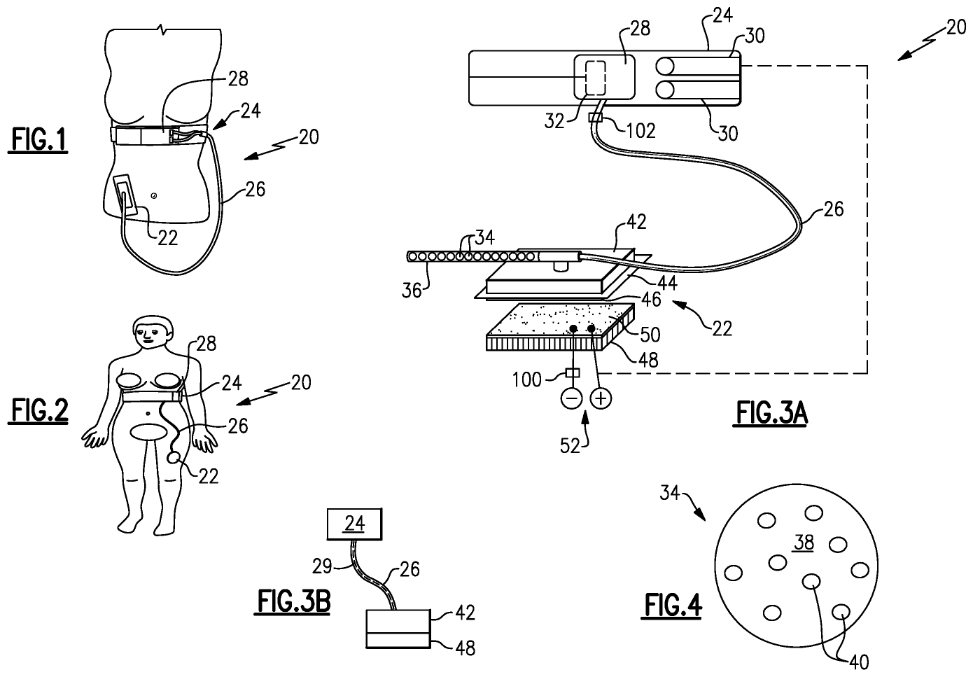

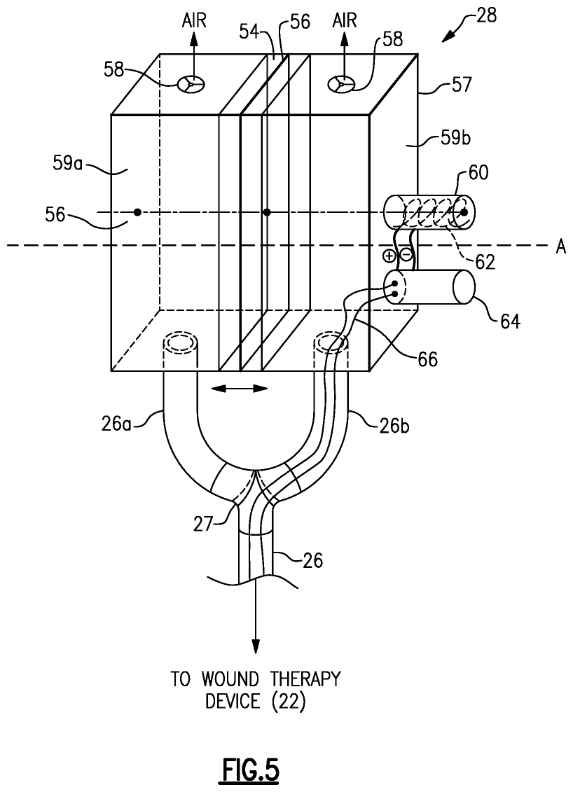

FIGS. 1-3B show a wound therapy system 20. The wound therapy system 20 includes a wound therapy device 22 and a wearable pump 28. In one example, the pump 28 is the pump described in U.S. patent application Ser. No. 14/676,221 (published as U.S. Patent Publication No. 2016/0287768) which is herein incorporated by reference in its entirety. A pneumatic tube 26 connects the wound therapy device 22 to the wearable pump 28. The wound therapy device 22 includes a plenum 42 and a porous body 48, which distributes vacuum provided by the wearable pump 28 over a wound site, and collects and transports fluids away from the wound site.

In one example, the pump is on a chest wrap 24. The chest wrap 24 includes one or more lines 30 wrapped on the chest wrap 24. In one example, the lines 30 are arranged in a serpentine pattern around the chest wrap 24. The lines 30 are connected to the pump 28. As the chest of a patient expands and contracts, the lines 30 translate the circumferential expansion-contraction of the chest into linear movement via an energy conversion device 32, which drives the pump 28. In one example, the lines 30 amplify the circumferential movement of the chest more than 1.2 times. The pump 28 creates a vacuum in the pneumatic tube 26. The pump 28 is dual-acting such that the vacuum is maintained continuously through both expansion and contraction of the chest (i.e., both inhaling and exhaling). In one variation, the lines 30 directly mechanically drive the pump 28, without the device 32.

In one example, at least a portion of the pneumatic tube 26 contains an absorbent and/or adsorbent media 34 (hereinafter “absorbent”), which in one example may be a plurality of balls supplied to the pneumatic tube 26 from a storage portion 36. As shown schematically in FIG. 4, a representative pellet of the absorbent media 34 has an outer, impermeable shell 38. The outer shell 38 contains perforations 40. Inside the outer shell 38 is an absorbent/adsorbent material such as a powder, which can retain fluid via the perforations 40. In one example, the powder consolidates or agglomerates upon contact with fluid without blocking the entire pneumatic tube 26.

In another example, the pneumatic tube 26 also includes a shapeable wire 29 allowing the patient to shape the pneumatic tube 26 for improved comfort. In yet another example, the pneumatic tube has a control switch 102.

The wound therapy device 22 is applied to a wound site of a patient. The wound therapy device 22 includes the plenum 42 and the porous body 48. In one example, the porous body 48 is a foam material. In this case, the plenum 42 is mounted on an adhesive dressing 44. At least one of the plenum 42 and adhesive dressing 44 is magnetic and has a magnetic field at surface 46. The porous body 48 is also magnetic and has a magnetic field at surface 50. The porous body 48 is adapted to be placed into or on a wound cavity. The porous body 48 is segmented, as will be discussed in more detail below. The magnetic surfaces 46, 50 attract and seal to one another such that the pneumatic tube 26 applies vacuum to the porous body 48 via the plenum 42. In one example, the magnetic seal is weak enough that the plenum 42 can be easily repositioned manually across the porous body 48 to concentrate vacuum in specific areas of the porous body 48.

The adhesive dressing 44 has a surface area that overhangs the magnetic surface 46 and is applied to skin surrounding the wound of the patient to secure and seal the wound therapy device 22 to the wound site. The adhesive dressing 44 also helps prevent infection to surrounding health skin by sealing off the wound site.

In one example, the porous body 48 has embedded conductive fibers and includes electrical leads 52 to provide electrical impulses to the conductive fibers. The electrical leads 52 have a control switch 100. These electrical impulses transmit electrical stimulation directly to the wound for pain management and stimulating healing. In a particular example, during removal of the wound therapy device 22, high voltage stimulation is provided to the electrical leads 52 to create a numbing effect for painless porous body 48 removal. In another example, the electrical leads 52 are connected to the chest wrap 24, which provides electrical power to the leads 52 generated by expansion and contraction of the patient's chest, similar to the description above with respect to the pump 28. The chest wrap 24 can also provide a grounding point for the leads 52.

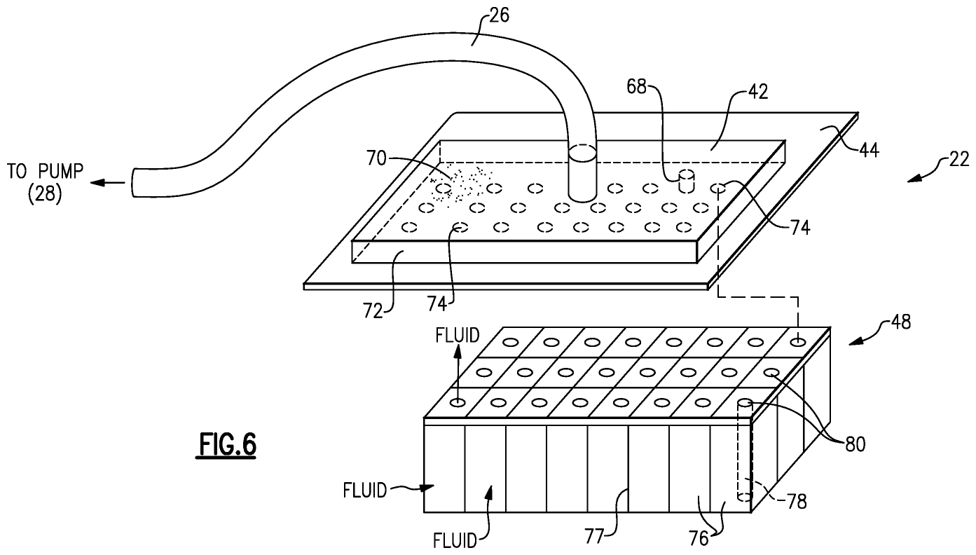

Referring now to FIG. 5, one example of the pump 28 is shown in more detail. The pump 28 includes a piston 54 movable along an axis A and dividing the pump into multiple chambers shown as 59a, 59b. Seals 56 are arranged around the periphery of the piston 54 dynamically sealing to the housing 57. One way valves 58 expel air from the pump 28 as the piston 54 changes the volumes of the chambers 59a, 59b. This generates a vacuum in ports 26a, 26b of the pneumatic tube, which correspond to the chambers 59a, 59b and which meet at a joint 27 to form the pneumatic tube 26. The pump 28 also includes an energy storage device 60 which stores energy collected from the chest wrap 24 as discussed above. The pump 28 also includes an electromechanical power generator 62 to convert the energy collected from the chest wrap 24 into electromechanical energy. The electromechanical power generator 62 provides power to a circuit or capacitor 64, which provides power to electrical leads 66 that are operably connected to the electrical leads 52 in the porous body 48 of the wound therapy device 22. The expelled air through valve 58 can be used by the system for other forms of useful work like inflating a catheter balloon or operating a CPAP machine.

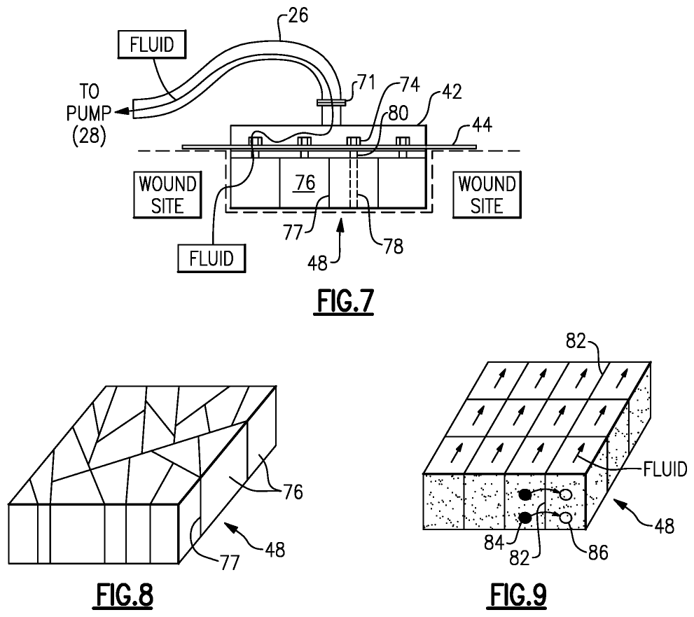

Referring now to FIGS. 6-7, an example of the wound therapy device 22 is shown in more detail. Here, the plenum 42 includes one or more raised bosses 68 to prevent backflow of fluid exiting the wound therapy device 22 back to the porous body 48. In another example, the plenum 42 can include an absorbent material 70, to absorb fluids from the wound. In yet another example, the pneumatic tube 26 connects to the plenum 42 with a removable lock 71 like a medical quick-disconnect.

A bottom surface 72 of the plenum 42 and the adhesive dressing 44 include holes 74 therethrough. The porous body 48 includes a plurality of segments 76 separated by dividers 77, and each segment includes a capillary 78. The dividers 77 are fluid impermeable (e.g., a polymer wall) and fluidly separate the segments 76. An outlet 80 from each capillary 78 aligns with a hole 74 in the plenum and adhesive dressing 44. A combination of vacuum from the pump 28 and capillary action draws fluid from the wound site through the porous body 48 and up into the plenum 42. The fluid is then absorbed by the absorbent material 70 in the plenum 42 and/or the absorbent material 34 in the pneumatic tube 26.

Though in the example shown in FIG. 6 each segment 76 has one capillary 78, in other examples each segment 76 has multiple capillaries 78. In other examples, the capillaries 78 can be vertical, horizontal, or oblique in other, random directions. Furthermore, the segments 76 can be uniform in size and shape as shown in FIGS. 6-7. Alternatively, the segments 76 are or random in size and shape, as shown in FIG. 8. The capillary 78 tube shape provides fluid-tube surface tension lift, assisting the removal of fluid from the wound site.

The segmentation of the porous body 48 prevents fluid from crossing into adjacent segments 76, which prevents cross-contamination at the wound site and directs fluid towards the plenum 28 to be removed from the wound site. Random segmentation of the porous body 48 aids in wound healing because when the dressing (i.e., porous body 48) is changed at the wound site, the new porous body 48 will have a new random segmentation and/or capillary pattern and will draw fluid from different areas of the wound. The size and shape of the segments 76 may be irregular or non-uniform. In one example, the segments sizes and shapes are randomly generated. The segmentations also improve the mechanical properties, such as resistance to crushing, of the porous body 48, in one example. The randomness of the size and shape of the segments 76 may be generated by computer or human in a processing stage of the porous body 42.

In another embodiment, shown in FIG. 9, the porous body 48 includes barrier dividers 82. The barrier dividers 82 include or are made from an anti-bacterial or anti-viral agent, such as silver. The barrier dividers 82 may be fluid permeable such that bacteria or viruses that crosses the barrier dividers 82 are killed.

Although a combination of features is shown in the illustrated examples, not all of them need to be combined to realize the benefits of various embodiments of this disclosure. In other words, a system designed according to an embodiment of this disclosure will not necessarily include all of the features shown in any one of the figures or all of the portions schematically shown in the figures. Moreover, selected features of one example embodiment may be combined with selected features of other example embodiments.

The preceding description is exemplary rather than limiting in nature.

Variations and modifications to the disclosed examples may become apparent to those skilled in the art that do not necessarily depart from this disclosure. The scope of legal protection given to this disclosure can only be determined by studying the following claims.

What is claimed is:

1. A wound therapy system, comprising:
a wound therapy comprising a porous body operable to carry a fluid from a wound site;
a tube; and
a pump connected to the porous body by the tube, the pump operable to apply vacuum through the tube to draw fluid from the wound site.

2. The wound therapy system of claim 1, further comprising a chest wrap, the chest wrap configured to transform circumferential expansion and contraction of the chest wrap into energy for driving the pump.

3. The wound therapy system of claim 2, wherein the chest wrap further comprises at least one serpentine line that amplifies power generation from circumferential expansion and contraction of the chest wrap.

4. The wound therapy system of claim 1, wherein the porous body includes capillaries operable to carry the fluid through the porous body.

5. The wound therapy system of claim 1, wherein the porous body includes a plurality of segments.

6. The wound therapy system of claim 5, wherein the plurality of segments are irregular in size, shape, or both.

7. The wound therapy system of claim 5, further comprising a plurality of dividers separating the plurality of segments from one another.

8. The wound therapy system of claim 7, wherein the plurality of dividers are fluid-impermeable.

9. The wound therapy system of claim 7, wherein the plurality of dividers include at least one of an anti-bacterial and an anti-viral agent.

10. The wound therapy system of claim 1, wherein the porous body includes electrically conductive fibers.

11. The wound therapy system of claim 10, further comprising electrical leads operable to provide electrical impulses to the conductive fibers.

12. The wound therapy system of claim 11, wherein the electrical leads are connected to and draw power from the chest wrap.

13. The wound therapy system of claim 1, wherein the tube includes an absorbent material operable to absorb the fluid.

14. The wound therapy system of claim 13, wherein the absorbent material comprises a plurality of balls, the balls including an impermeable shell having perforations, and an absorbent powder inside the impermeable shell.

15. The wound therapy system of claim 13, wherein the absorbent material solidifies upon contact with the fluid.

16. The wound therapy system of claim 1, wherein the wound therapy device further includes a plenum, the plenum in fluid communication with the tube.

17. The wound therapy system of claim 16, further comprising an adhesive dressing between the plenum and the porous body.

18. The wound therapy system of claim 17, wherein the adhesive dressing seals the wound site.

19. The wound therapy system of claim 17, wherein at least one of the plenum and adhesive dressing are magnetic, and the porous body is magnetic, and the plenum or adhesive surface and porous body attract and seal to one another.

20. A wound therapy system, comprising:
a wound therapy device comprising a foam wound dressing having a plurality of segments, the foam including capillaries operable to carry fluid from a wound site;
a pneumatic tube in fluid communication with the wound therapy device; and
a pump operable to apply vacuum to the foam via the pneumatic tube to draw fluid out of the wound site.
